(19) United States
(12) Patent Application Publication
(10) Pub. No.: US 2006/0019401 A1
(43) Pub. Date: Jan. 26, 2006

(54) Monitoring the stability of vinylog compounds

(76) Inventors: Matthias Fies, Krefeld (DE); Ronald Klagge, Erkrath (DE); Jan Mirco Stachowiak, Essen (DE)

Correspondence Address: COGNIS CORPORATION;PATENT DEPARTMENT, 300 BROOKSIDE AVENUE, AMBLER, PA, 19002, US

(21) Appl. No.: 10/523,278

(22) PCT Filed: Jul. 24, 2003
(86) PCT No.: PCT/EP03/08105

§ 371 Date: Feb. 1, 2005

(30) Foreign Application Priority Data
Aug. 2, 2002 (DE) 102 35 643.2

(51) Int. Cl. G01N 33/00

(52) U.S. Cl. 436/129

(57) ABSTRACT

The process is used to monitor the stability of compositions and reaction mixtures which contain vinylog compounds, more especially (meth)acrylic acid and/or (meth)acrylates. The content of dissolved oxygen in the composition or in the reaction mixture is determined and compared with predetermined reference values. Incipient or threatening polymerization is detected as early as possible and well before polymerization actually begins. Monitoring can be carried out simply, inexpensively and, in particular, safely both during storage and transportation and during the course of reactions, more particularly during the esterification of (meth)acrylic acid with mono- or polyhydric alcohols.

This invention relates to a process for monitoring the stability of compositions and reaction mixtures which contain vinylog compounds, more especially (meth)acrylic acid and/or (meth)acrylates. In the following, the terms (meth)acrylic acid and (meth)acrylates encompass acrylic acid and/or methacrylic acid and acrylates and/or methacrylates, respectively. The stability of these compositions and reaction mixtures to polymerization is to be monitored.

In the following, the invention is explained in connection with (meth)acrylic acid and (meth)acrylates although the invention is not limited to these substances and may be applied to any vinylog compounds where the same or similar problems arise.

The monitoring process according to the invention may be used with particular advantage in the production of esters of acrylic acid and/or methacrylic acid with mono- or polyhydric alcohols by reaction of the reactants in the presence of acidic esterification catalysts and polymerization inhibitors and during the storage and transportation of the starting materials and the reaction products.

The storage and reaction of (meth)acrylic acid and (meth)acrylates in large storage tanks and reaction vessels is problematical on account of the tendency of these compounds to polymerize. Unwanted and, generally, uncontrolled polymerization not only results in the loss of this raw material, the exothermic nature and the resulting, possibly explosive course of the polymerization also represent a danger to people and objects present in the vicinity of the storage tank or reaction vessel.

In order to reduce the danger of unwanted polymerization, inhibitors are added to the (meth)acrylic acid or the (meth)acrylates and, under certain conditions, are capable of terminating any, generally radical polymerization reaction which may occur. The most commonly used inhibitor is hydroquinone monomethyl ether (MeHQ). In general, both individual compounds and several components from the class of alpha-substituted phenolic compounds may be used as polymerization inhibitors. Examples include comparatively low volatility compounds based on correspondingly substituted monohydric or polyhydric phenols, dihydric phenols of the disubstituted hydroquinone derivative type being particularly suitable as polyhydric phenolic compounds. Other examples are p-methoxyphenol, 2,5-di-tert.-butyl-p-cresol and/or tert.butyl pyrocatechol and 2,5-di-tert.butyl hydroquinone.

The polymerization inhibitor or optionally the inhibitor mixture is added to the reaction mixture in quantities of normally 200 to 10,000 ppm and preferably ca. 300 to 2,000 ppm. These figures are based on the weight of the reaction mixture consisting of (meth)acrylic acid and polyhydric alcohols.

Examples of polyalcohols to be esterified include ethylene glycol, propylene glycol, butane-1,4-diol, hexane-1,6-diol, decane-1,10-diol, dimer diol, for example “Sovermol 908” (Cognis), neopentyl glycol, diethylene glycol, triethylene glycol, dimethylol propane, glycerol, trimethylolpropane, trimethylolhexane, trimethylolethane, hexane-1,3,5-triol and pentaerythritol.

Despite the presence of inhibitors, however, stored (meth)acrylic acid and (meth)acrylates have to be constantly monitored to enable any polymerization reaction that may begin despite inhibition to be detected as quickly as possible and the necessary counter-measures to be initiated.

In practice, various known processes are available for this purpose.

The most commonly used process is based on monitoring of the temperature of the stored (meth)acrylic acid or (meth)acrylates. Any polymerization occurring is detected by utilizing the effect whereby the heat of reaction released by the polymerization leads to an increase in the temperature of the stored material. By using temperature sensors in the storage tank, such increases in temperature can be recorded and any incipient polymerization can be detected. This process is described in the company brochure “Acrylic Acid—A Summary of Safety and Handling”, Rohm & Haas Company, 3rd Edition, page 13.

Another process comprises measuring the extinction of the stored material in relation to visible light. This process is based on the effect that poly(meth)acrylic acid and poly(meth)acrylates are insoluble in the respective monomers and therefore cause clouding of the stored material or lead to a change in the refractive index. Accordingly, suitable optical sensors are capable of detecting any polymerization already in progress.

In another process, polymerization is detected through the increase in viscosity. This increase can be detected, for example, through the increased energy input of a stirrer mixing the liquid which can be measured through the increased power consumption of the stirrer motor.

Unfortunately, the processes mentioned above are attended by the major disadvantage that it is generally not possible to save the material affected by polymerization by suitable counter-measures.

This is because the sensors used in the prior art only respond when polymerization has already started and a large quantity of monomers has already been reacted to form the polymer because such effects as increases in temperature and clouding can only be measured then. The same applies to monitoring of the viscosity.

Since it is generally not possible to use partly polymerized material in the production process or to sell it on the market as a product, the affected material is lost from the economic perspective.

It is therefore desirable to be able to detect polymerization as early as possible. Ideally, certain conditions which can lead to polymerization should be detected even before the actual polymerization begins. Only in this case is it possible to usefully save the affected material.

These considerations apply not only to the storage and transportation of vinylog compounds, but increasingly to reactions, more especially for the production of esters of (meth)acrylic acid with mono- or polyhydric alcohols and/or epoxides by esterification, transesterification or ring-opening addition of (meth)acrylic acid onto terminal or internal epoxides. In esterification, the reactants are reacted in the presence of acidic esterification catalysts at elevated temperature and optionally reduced pressure to form the esters. On the one hand, the reaction mixture is destabilized by the high temperature so that polymerization starts earlier. On the other hand, it is known that the polymerization inhibitors normally used are activated by the free oxygen dissolved in the reaction mixture. However, under reduced pressure, which is necessary for removing the water of esterification from the circuit, the content of dissolved free oxygen decreases so that for this reason, too, destabilization occurs earlier than in the case of vinylog compounds which are stored at low temperatures and under normal pressure. Measures for monitoring and preventing polymerization during such reactions are therefore of particular importance.

Accordingly, the problem addressed by the present invention was to detect incipient or threatening polymerization as early as possible and well before polymerization actually begins in the monitoring process mentioned at the beginning. Monitoring would be able to be carried out simply, inexpensively and, in particular, safely both during storage and transportation and during reactions, more particularly during the esterification of (meth)acrylic acid with mono- or polyhydric alcohols.

In the process mentioned at the beginning for monitoring the stability of compositions and reaction mixtures containing vinylog compounds, the solution to this problem as provided by the invention is characterized in that the content of dissolved oxygen in the composition or in the reaction mixture is determined and compared with predetermined reference values.

For certain concentrations of dissolved oxygen under the predetermined conditions, such as for example and in particular at certain temperatures, the reference values indicate how safe the condition of the monitored system is with respect to polymerization and how far away the actual condition of the system is from conditions which represent a serious danger of polymerization. Thus, the danger of polymerization is particularly high if no, or hardly any, dissolved oxygen can be detected in the monitored system. On the other hand, the monitored system is safer and more stable, the greater the amount of dissolved oxygen detected in the monitored system.

The process according to the invention offers a simple way of detecting conditions that could lead to polymerization of (meth)acrylic acid or (meth)acrylates or other vinylog compounds during their storage or chemical reaction and thus enables the user to initiate counter-measures even before polymerization actually begins. The process according to the invention makes use of the fact that the polymerization inhibitor can only develop its inhibiting effect when, at the same time, a sufficient quantity of molecular oxygen is dissolved in the material to be stabilized. In fact, the deficiency of dissolved oxygen is the most frequent cause of unwanted polymerization despite an adequate content of polymerization inhibitor.

In the process according to the invention, therefore, it is essential to measure the content of molecular oxygen dissolved in the particular vinylog material, more especially (meth)acrylic material, and to assess the risk of polymerization by subsequent comparison with a reference value and optionally to initiate counter-measures.

As already mentioned, the process according to the invention may be used not only for the storage and transportation but also in ongoing reactions and is particularly suitable for reactions which take place under reduced pressure because, in their case, the oxygen content in the reaction mixture is generally greatly reduced so that there is an increased risk of polymerization.

In a particularly preferred embodiment of the process according to the invention, the time required for complete consumption of the dissolved oxygen is determined from the measured content of dissolved oxygen and the rate at which oxygen is consumed under the particular conditions, more especially the particular temperature. The time determined in this way indicates the time frame in which counter-measures need to be taken, for example in the form of the introduction of oxygen or air or an increase in the throughflow rate of those gases. At the end of this period at the latest, there is a high risk of polymerization beginning—in the worst case explosively. If monitoring is undertaken during an ongoing reaction, it is possible by comparing the established time required for complete consumption of the dissolved oxygen with the time required for completion of the reaction to determine whether any counter-measures need to be taken at all or whether the oxygen already present will last until the end of the reaction.

In another advantageous embodiment, dangerous changes in the tendency towards polymerization can be immediately detected without delay by continuously determining the dissolved oxygen content and comparing the content determined with reference values, again continuously. If the comparison shows that the situation is dangerous, an automatic warning signal (visual and/or acoustic) may advantageously be released and the corresponding appropriate counter-measure can be initiated, more especially automatically. This measure may take the form of, for example, an increase in the supply of air or oxygen.

As already mentioned, the process according to the invention is used with particular advantage in ongoing reactions carried out in particular under reduced pressure because, in this case, only relatively little oxygen can be dissolved in the reaction mixture so that there is an increased risk of polymerization.

In the process according to the invention, the dissolved oxygen content can be determined in various ways. It can be measured with a suitable oxygen sensor. In this case, the dissolved oxygen content can be measured with an amperometric sensor. An amperometric sensor is understood to be a ready-to-use measuring cell which is used for measuring the concentration of cathodically reducible or anodically oxidizable chemical compounds. For example, amperometric oxygen sensors are known.

The dissolved oxygen content can also be measured by titration. Finally, the dissolved oxygen content can be measured by spectroscopic methods, more particularly in the IR or NIR spectral region.

According to the invention, the dissolved oxygen content may also be determined in the composition to be investigated or in the reaction vessel. Alternatively or additionally, part of the composition or the reaction mixture may be removed, more especially continuously, from the reaction vessel, passed through a measuring cell where the dissolved oxygen content is determined and preferably returned to the reaction vessel.

In another particularly advantageous embodiment, the dissolved oxygen content is determined at several different places within the composition or within the reaction mixture. This is because the oxygen content is generally not the same throughout the composition or the reaction vessel. In the upper region, the particularly low pressure leads, for example, to a reduced content of dissolved gases and also oxygen. In the lower part of the vessel, the oxygen content is generally higher on account of the higher pressure. However, if nevertheless air or another oxygen mixture is introduced into the lower part of the vessel, as is normally the case, the oxygen content in the vicinity of these nozzles may not be as high as assumed due to the prevailing pressure because the gas still had too little time to dissolve. For these reasons, it is of advantage to determine the oxygen content at different places. A local reduction in the dissolved oxygen content can also be dangerous in regard to polymerization. In another embodiment, therefore, the dissolved oxygen content is determined in the upper region of the liquid phase of the composition or the reaction mixture. Accordingly, it is of advantage to determine the oxygen content in the lower region.

The inflammability and possible danger of explosion of acrylic acid vapors in the presence of oxygen above the liquid level of the composition or the reaction mixture make it appear advisable to work with relatively low free oxygen contents in the liquid phase in order to establish a correspondingly low oxygen content in the gas phase. However, this requirement is at variance with the goal of establishing a high concentration of free oxygen in the liquid phase for stabilization purposes. Accordingly, it is advisable carefully to co-ordinate the oxygen content in the gas phase with the oxygen content in the liquid phase in order to establish optimal working conditions which take both requirements sufficiently into account. To this end, it is of advantage if, in addition to the oxygen content in the liquid phase, the oxygen content above the liquid phase is also determined, more especially by means of a sensor. The oxygen content in the system may then readily be adjusted in such a way that, on the one hand, the liquid phase is safe against polymerization in the liquid phase and, on the other hand, the oxygen content in the gas phase remains below the explosion limit.

Finally, in another embodiment, the monitoring process according to the invention is carried out during the production of (meth)acrylic acid esters of mono- or polyhydric alcohols by esterification of the reactants, more especially under reduced pressure.

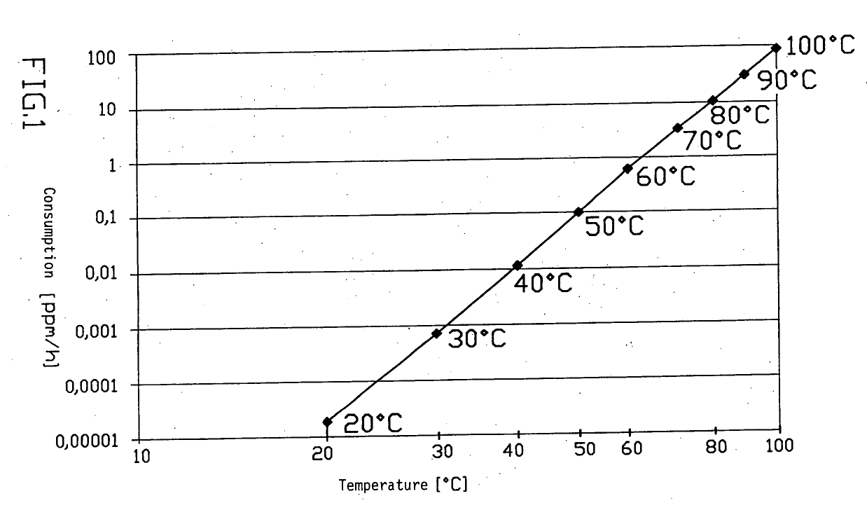

One example of the predetermined reference values with which the measured dissolved oxygen content can be compared is shown in FIG. 1 where the consumption of oxygen dissolved in acrylic acid in ppm per hour is plotted against various temperatures in ° C. with logarithmic scale on both axes, the starting concentration being taken as 50 ppm. If the dissolved oxygen content in acrylic acid is determined in accordance with the invention, the oxygen consumption can be read off with these values at the particular temperature of the acrylic acid, so that it is possible to calculate the time after which—without any further input of oxygen—no more oxygen is present in the liquid acrylic acid. This period on the one hand is a measure of the safety level against unwanted polymerization and, on the other hand, indicates the time frame in which counter-measures need to be taken.

The monitoring process according to the invention can be used with advantage not only during the reaction of (meth)acrylic acid with the mono- or polyhydric alcohols, but also for the storage of (meth)acrylic acid and for the storage and transportation of the product produced, i.e. the (meth)acrylic acid ester. In order always reliably to prevent polymerization, the dissolved oxygen content should not fall below 5 ppm.

During the reaction of the acrylic acid with the mono- or polyhydric alcohol, air or another oxygen-containing gas is passed through the liquid reaction mixture in known manner in the form of small bubbles. Even when the reaction is over, this gas should continue to be bubbled through the reaction mixture in order to prevent polymerization. Using the monitoring process according to the invention, it is possible to determine when bubbling through of the gas can be stopped. In this way, not too much oxygen is introduced into the product so that product damage in the form of discoloration can be avoided with a high level of reliability.

Even during storage of the starting product or end product, it is advisable to measure the oxygen content by the process according to the invention to ascertain when more air needs to be introduced. This provides for considerably safer and yet more economical handling than in the prior art where the main focus is solely the change in temperature.

### EXAMPLES

### Example 1

### Solventless Esterification

779.8 g of acrylic acid (Merck, Hohenbrunn), 760.5 g of ethoxylated trimethylolpropane (OH value 680 mg KOH/g; Perstorp, Sweden), 53.9 g of p-toluenesulfonic acid (Sigma-Aldrich, Deisenhofen) as catalyst and 2.48 g of ditert.butyl hydroquinone (Sigma-Aldrich) as inhibitor were reacted in a 2-liter flask.

During the esterification reaction, air was passed through the reaction mixture (25 l/h) and water was removed. At a temperature of 75° C. and a pressure p falling from 125 to 10 hPa during the reaction, the reaction time was 10 hours. A crude product with the following properties was obtained:

| Acid value: | 18 mg KOH/g |
|---|---|
| OH value: | 23 mg KOH/g |
| Gardner color value: | 4 |
| Water content: | 0.1% |

The dissolved oxygen content potentiometrically determined during the reaction is shown in Table 1 below as a function of the pressure:

TABLE 1

| | p [hPa] | | | | | | |
|---|---|---|---|---|---|---|---|
| | 125 | 100 | 80 | 60 | 40 | 25 | 10 |
| O₂ [ppm] | 21 | 13 | 12 | 10 | 8 | 6 | 5 |

For security against unwanted polymerization, it was determined in advance that the oxygen content should not fall below 5 ppm. Accordingly, when the oxygen content had fallen to 5 ppm at a pressure of 10 hPa, the pressure p was not reduced any further in order to stop the oxygen content from falling below the limit mentioned.

### Example 2

### Esterification in the Presence of a Solvent

532.6 g of ethoxylated trimethylolpropane (OH value 680 mg KOH/g; Perstorp), 557.8 g of acrylic acid (Merck), 437.7 g of methyl cyclohexane (Merck), 4.73 g of sulfuric acid (Merck) and 2.93 g of hydroquinone monomethyl ether (Merck) were heated to boiling temperature in a 2-liter flask. An air stream of 25 l/h was passed through the reaction mixture. The water formed during the reaction was azeotropically distilled off. After a reaction time of 11 hours, the solvent was distilled off under reduced pressure.

The crude product obtained in this way had the following properties:

| Acid value: | 11 mg KOH/g |
|---|---|
| OH value: | 25 mg KOH/g |
| Gardner color value: | 2 |
| Water content: | 0.2% |

The dissolved oxygen content potentiometrically determined repeatedly during the reaction is shown in Table 2 below as a function of the reaction time:

TABLE 2

| | t [h] | | | | | | |
|---|---|---|---|---|---|---|---|
| | 0 | 2 | 4 | 6 | 8 | 10 | 11 |
| O₂ [ppm] | 40 | 43 | 45 | 46 | 46 | 47 | 47 |

The measured values show that the reaction was carried out entirely in the safe range so that there was no need for special measures, such as increasing the air throughflow rate.

1-15. (canceled)

16. A process for monitoring the stability of compositions which contain vinylog compounds, which comprises: (a) determining a content of dissolved oxygen in the composition; and (b) comparing the content of dissolved oxygen in the composition with predetermined reference values for a condition of the composition, whereby, the stability of the composition is determined.

17. The process as claimed in claim 16, wherein, the stability is monitored by determining a time required for complete consumption of the dissolved oxygen from the measured content of dissolved oxygen and the rate at which oxygen is consumed under the conditions of the composition.

18. The process as claimed in claim 16, wherein, the dissolved oxygen content is continuously determined and comparison of the dissolved oxygen content determined with the reference values is continuously carried out.

19. The process as claimed in claim 16, wherein, the composition comprises a reacting mixture under reduced pressure.

20. The process as claimed in claim 16,wherein the dissolved oxygen content is measured with an oxygen sensor.

21. The process as claimed in claim 16, wherein, the dissolved oxygen content is amperometrically determined.

22. The process as claimed in claim 16, wherein, the dissolved oxygen content is determined by titration.

23. The process as claimed in claim 16, wherein, the dissolved oxygen content is determined by spectroscopic methods, in at least one of an IR and an NIR spectral region.

24. The process as claimed in claim 16, wherein, the dissolved oxygen content is determined in the composition contained in a vessel selected from the group consisting of the reaction vessels, storage vessels and transportation vessels.

25. The process as claimed in claim 16, wherein, a portion of the composition being monitored is removed from a vessel, passed through an analytical device where the dissolved oxygen content is determined, and optionally returned to the vessel.

26. The process as claimed in claim 16, wherein, the dissolved oxygen content is determined at several different locations within the composition contained in a vessel.

27. The process as claimed in claim 16, wherein, the dissolved oxygen content is determined in an upper region of a liquid phase of the composition.

28. The process as claimed in claim 16, wherein, the dissolved oxygen content is determined in a lower region of a liquid phase of the composition.

29. The process as claimed in claim 16, wherein, an oxygen content is additionally determined in a vapor phase above a liquid phase in a vessel by means of a sensor.

30. The process as claimed in claim 16, wherein, the composition of which the stability is monitored, comprises a reacting mixture for production of (meth)acrylic acid esters of mono- or polyhydric alcohols, the reacting mixture comprising (meth)acrylic acid esters, wherein, the reacting mixture is optionally under reduced pressure.

31. The process as claimed in claim 17, wherein, the dissolved oxygen content is continuously determined and comparison of the dissolved oxygen content determined with the reference values is continuously carried out.

32. The process as claimed in claim 17, wherein, the composition comprises a reacting mixture under reduced pressure.

33. The process as claimed in claim 17, wherein, the dissolved oxygen content is determined in the composition contained in a vessel selected from the group consisting of the reaction vessels, storage vessels and transportation vessels.

34. The process as claimed in claim 24, wherein, a portion of the composition being monitored is removed from a vessel, passed through an analytical device where the dissolved oxygen content is determined, and optionally returned to the vessel.
